(19) United States
(12) Patent Application Publication
(10) Pub. No.: US 2019/0184085 A1
(43) Pub. Date: Jun. 20, 2019

(54) END CAP OF DIALYZER AND FABRICATING METHOD THEREOF, AND DIALYZER

(71) Applicants: LITE-ON ELECTRONICS (GUANGZHOU) LIMITED, GUANGZHOU (CN); Lite-On Technology Corporation, Taipei (TW)

(72) Inventors: Wei-Ming Shieh, Taipei (TW); Yi-Feng Pu, Taipei (TW); Pei-Hsuan Huang, Taipei (TW)

(73) Assignees: LITE-ON ELECTRONICS (GUANGZHOU) LIMITED, GUANGZHOU (CN); Lite-On Technology Corporation, Taipei (TW)

(21) Appl. No.: 16/223,134

(22) Filed: Dec. 18, 2018

(30) Foreign Application Priority Data
Dec. 18, 2017 (CN) 201711362480.6

(51) Int. Cl. A61M 1/16

(52) U.S. Cl. CPC A61M 1/1623; A61M 1/1654; A61M 1/1652; A61M 1/1601

(57) ABSTRACT

An end cap of a dialyzer and a fabricating method thereof, and a dialyzer are provided. The end cap includes a main body and a sealing element. The main body has a blood port. The sealing element is integrally connected on an inner wall of the main body.

### CROSS REFERENCE TO RELATED APPLICATION

This application claims the priority benefit of China application serial no. 201711362480.6, filed on Dec. 18, 2017. The entirety of the above-mentioned patent application is hereby incorporated by reference herein and made a part of specification.

### BACKGROUND OF THE INVENTION

### Field of the Invention

The invention relates to a dialyzer, and more particularly, to an end cap of a dialyzer and a fabricating method thereof.

### Description of Related Art

Patients with renal failure would experience their kidney cannot filter waste accumulating in their body, such as protein-digested products, urea, creatinine, phosphate, or vitamin B12, and therefore require dialysis to compensate for the natural excretory function of the kidneys. A common dialysis includes, for instance, purifying the blood of the patients using a dialyzer to remove excess water and toxins from the blood.

### SUMMARY OF THE INVENTION

The invention provides an end cap of a dialyzer and a fabricating method thereof, thereby effectively enabling reduction of production process and processing time.

The invention provides a dialyzer having improved liquid tightness.

The invention provides an end cap of a dialyzer including a main body and a sealing element. The main body has a blood port. The sealing element is integrally connected on an inner wall of the main body.

According to an embodiment of the invention, in the end cap of the dialyzer, the inner wall of the main body can have a notch, a latch, a snap, or an inner thread.

According to an embodiment of the invention, in the end cap of the dialyzer, the material of the main body is, for instance, polyvinylchloride (PVC), polymethylmethacrylate (PMMA), polyethylene terephthalate (PET), polycarbonate (PC), polypropylene (PP), polysulfone (PSU), polyethylene (PE), polyacrylonitrile (PAN), Nylon (such as Nylon 6), acrylonitrile butadiene styrene (ABS), or a combination thereof.

According to an embodiment of the invention, in the end cap of the dialyzer, the material of the sealing element is, for instance, a rubber or an elastomer.

According to an embodiment of the invention, in the end cap of the dialyzer, the elastomer is, for instance, a thermoplastic elastomer (TPE) or a thermoset elastomer.

According to an embodiment of the invention, in the end cap of the dialyzer, the main body and the sealing element are integrally connected via, for instance, a double injection molding method.

According to an embodiment of the invention, in the end cap of the dialyzer, the shrinkage rate difference between the main body and the sealing element is, for instance, 1% or less.

The invention provides a fabricating method of an end cap of a dialyzer including the following steps. The main body and the sealing element are integrally formed using a double injection molding method. The main body has a blood port. The sealing element is integrally connected on an inner wall of the main body.

According to an embodiment of the invention, in the fabricating method of the end cap of the dialyzer, the main body can be injection-molded first, and then the sealing element is injection-molded. The softening point of the main body is, for instance, higher than the softening point of the sealing element.

According to an embodiment of the invention, in the fabricating method of the end cap of the dialyzer, the sealing element can be injection-molded first, and then the main body is injection-molded. The softening point of the sealing element is, for instance, higher than the softening point of the main body.

The invention provides a dialyzer including a housing, a sealant, a plurality of hollow fiber membranes, and two end caps. The housing has two openings opposite to each other. A dialysate inlet and a dialysate outlet are disposed on the housing. The sealant seals the two openings. The hollow fiber membranes disposed in the housing are secured by the sealant.

The end caps respectively cover two ends of the housing. Each of the end caps includes a main body and a sealing element. The main body has a blood port. The sealing element is integrally connected on an inner wall of the main body.

According to an embodiment of the invention, in the dialyzer, the material of the housing is, for instance, polypropylene (PP), polybutylene, polyethylene (PE), or a combination thereof.

According to an embodiment of the invention, in the dialyzer, the combining method of the housing and the end caps can include an engagement of corresponding recess and protrusion structures via jointing, clipping or screwing.

According to an embodiment of the invention, in the dialyzer, the outer wall of the housing can have a latch, a snap, a notch, or an external thread, and the inner wall of the main body can have a notch, a latch, a snap, or an inner thread correspondingly.

According to an embodiment of the invention, in the dialyzer, the combining method of the housing and the two end caps can further include performing ultrasonic welding after the jointing, clicking or screwing of the housing and the respective end caps.

According to an embodiment of the invention, in the dialyzer, the material of the main body is, for instance, polyvinylchloride (PVC), polymethylmethacrylate (PMMA), polyethylene terephthalate (PET), polycarbonate (PC), polypropylene (PP), polysulfone (PSU), polyethylene (PE), polyacrylonitrile (PAN), Nylon, acrylonitrile butadiene styrene (ABS), or a combination thereof.

According to an embodiment of the invention, in the dialyzer, the material of the sealing element is, for instance, a rubber or an elastomer.

According to an embodiment of the invention, in the dialyzer, the elastomer is, for instance, a thermoplastic elastomer (TPE) or a thermoset elastomer.

According to an embodiment of the invention, in the dialyzer, the forming method of the main body and the sealing element is, for instance, a double injection molding method.

According to an embodiment of the invention, in the dialyzer, the shrinkage rate difference between the main body and the sealing element is, for instance, 1% or less.

Based on the above, in the end cap of the dialyzer and the fabricating method thereof provided in the invention, the main body and the sealing element are integrally formed in a single piece. Therefore, the production process can be simplified, and production time can be effectively reduced. Moreover, in the dialyzer provided in the invention, since the sealing element is accurately positioned and integratedly secured on the main body beforehand, the dialyzer that includes the end caps and the housing combined together can have good liquid tightness. The conventional procedure of placing an additional sealing element in a structural gap or groove formed in an end cap is no longer required. As a result, the production steps and production time of the dialyzer can be effectively reduced.

In order to make the aforementioned features and advantages of the disclosure more comprehensible, embodiments accompanied with figures are described in detail below.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1A is a perspective view of an end cap of a dialyzer according to an embodiment of the invention.

FIG. 1B is a perspective view of the end cap of the dialyzer shown in FIG. 1A from another viewing angle.

FIG. 2 is a perspective view of a dialyzer according to an embodiment of the invention, of which a housing and end caps are not combined.

FIG. 3 is a perspective view of the dialyzer shown in FIG. 2, of which the housing and the end caps are combined.

### DESCRIPTION OF THE EMBODIMENTS

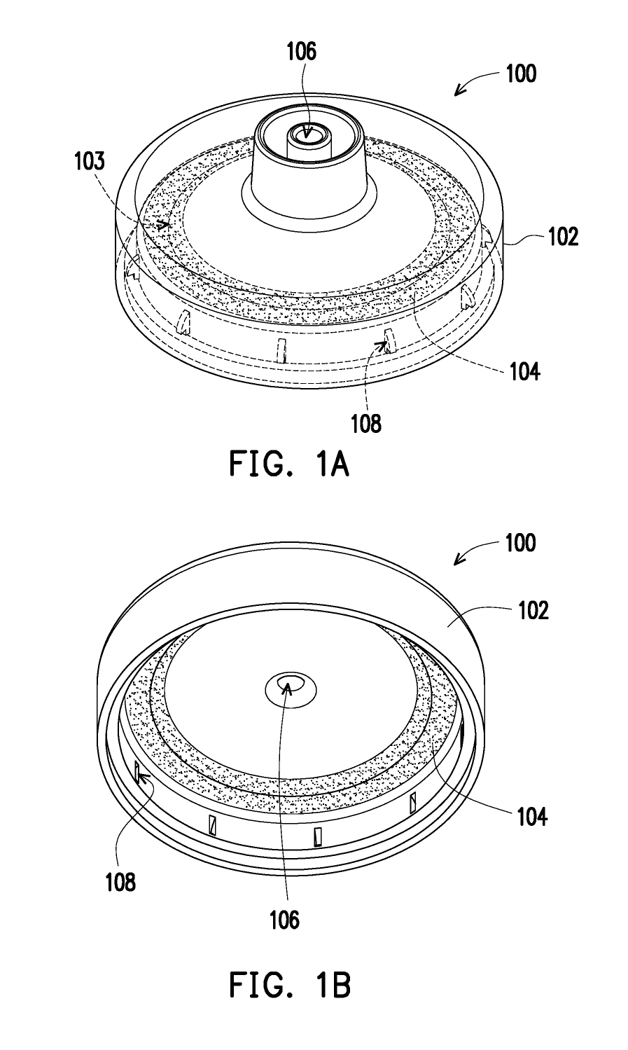

FIG. 1A is a perspective view of an end cap of a dialyzer according to an embodiment of the invention. FIG. 1B is a perspective view of the end cap of the dialyzer shown in FIG. 1A from another viewing angle.

Referring to both FIG. 1A and FIG. 1B, an end cap 100 of a dialyzer includes a main body 102 and a sealing element 104. Since the main body 102 and the sealing element 104 are integrally formed in a single piece, the production process of the end cap could be simplified, thereby effectively reducing the production time.

For instance, the end cap 100 can be fabricated via a double injection molding method to integrally form an integrated single-piece of the main body 102 and the sealing element 104. In the double injection molding process, the shrinkage rates of the selected materials are respectively between 0.2% and 5%, and the difference in the shrinkage rate (i.e., shrinkage rate difference) between the two materials respectively used in the first injection molding and the second injection molding can be 0% to 4.8%. In an embodiment, the difference in the shrinkage rate between the main body 102 and the sealing element 104 can be 1% or less, and further can be 0.6% or less, such as 0.4%, so as to avoid a decrease in interface strength of the main body 102 and the sealing element 104. During the double injection molding process, the materials of the main body 102 and the sealing element 104 undergo the respective injection molding, and the interface strength of the main body 102 and the sealing element 104 would be lessened with a huge difference in the shrinkage rate between the chosen materials. In the present embodiment, the definition of the shrinkage rate is the size difference between the mold cavity and the molded product at room temperature, which is then divided by the size of the mold cavity, wherein the shrinkage rate is defined by the thermal expansion and contraction as well as molding conditions of the materials themselves.

In the double injection molding process, the main body 102 can be injected first, and then the sealing element 104 is injected, wherein the softening point of the main body 102 might be higher than the softening point of the sealing element 104. Accordingly, the profile of the main body 102 injected first is not deformed because of a melting washout phenomenon. Moreover, the melting temperature of the main body 102 is, for instance, higher than the melting temperature of the sealing element 104. Specifically, the fabricating method of the end cap 100 can include, but not be limited thereto, the following steps. The material of the main body 102 is heated to a molten state, and the molten material is then injection-molded to form the main body 102. The main body 102 is placed in another mold, and a secondary injection molding is performed to form the sealing element 104. The secondary injection molding could be implemented by heating the material of the sealing element 104 to a molten state, and then injection-molding the molten material on the main body 102 and completing the engagement of the two materials at the same time. Therefore, a molten bonding layer 103 is further formed at the junction between the main body 102 and the sealing element 104. In an embodiment, the molten bonding layer 103 could be a heterojunction of the main body 102 and the sealing element 104, and the material thereof includes the material of the main body 102, the material of the sealing element 104, or a mixture thereof. The molten bonding layer 103, for instance, combines the main body 102 and the sealing element 104 via the viscosity of at least one of the molten materials or chemical bonding, so as to provide an integrated one-piece structure.

In another embodiment, in the double injection molding process, the sealing element 104 can be injected first, and then the main body 102 is injected, wherein the softening point of the sealing element 104 is, for instance, higher than the softening point of the main body 102. Therefore, the profile of the sealing element 104 injected first is not deformed by a melting washout phenomenon. Specifically, the fabricating method of the end cap 100 can include, but not be limited thereto, the following steps. The material of the sealing element 104 is heated to a molten state, and then the molten-state material is injection-molded to form the sealing element 104. The sealing element 104 is placed in another mold, and a secondary injection molding is performed to form the main body 102. The secondary injection molding could be implemented by heating the material of the main body 102 to a molten state, and then injection-molding the molten material on the sealing element 104 and completing the combination of the two materials at the same time. It is noted that the end cap 100 integrally formed in a single process, i.e., double injection molding, could facilitate the improved liquid tightness of the dialyzer using the same, while the production procedure and cost are reduced.

In contrast, in case a sealing ring, e.g., an O-ring, is not integrally formed with an end cap by double injection molding, the end cap would be designed to have an annular groove or a trapezoidal platform for accommodating the separate sealing ring to avoid blood leakage. The annular groove or trapezoidal platform is generally larger in dimensions than the O-ring to be placed thereinto, so that a gap exists between the end cap and the O-ring, thereby impacting the liquid tightness of the dialyzer. When the dialyzer having the end caps with the separate O-rings is utilized in hemodialysis, the ineffectiveness of the dialysis or poor dialyzing effect would occur owing to the deviation of the blood flow rate (QB) resulted from the varied pressure difference within the dialyzer. In addition, the arrangement of the separate O-rings would lead to extra space laid between the tubular housing and end caps of the dialyzer, and therefore, the yield would be reduced because of the increasing mis-positioning of the O-rings. Thus, the dialyzer without the integrally-formed end caps would suffer the poor dialyzing effect and lower liquid tightness.

Referring to FIG. 1A and FIG. 1B, the main body 102 has a blood port 106. The blood port 106 can be used as the blood input or blood output of the dialyzer. The inner wall of the main body 102 can have at least one notch, latch, snap (e.g., snap hook), or inner thread, each of which respectively functions as a recess or protrusion structure for the engagement. The main body 102 and the notch, the latch, the snap, or the inner thread can be integrally formed. The main body 102 can be combined with the tubular housing of the dialyzer via the engagement of the notch, the latch, the snap, or the inner thread on the inner wall of the main body 102 with the corresponding latch, snap, notch, or external thread on the outer wall of the housing. In the present embodiment, the inner wall of the main body 102 is exemplified by having a plurality of notches 108. The material of the main body 102 is, for instance, a hard material such as polyvinylchloride (PVC), polymethylmethacrylate (PMMA), polyethylene terephthalate (PET), polycarbonate (PC), polypropylene (PP), polysulfone (PSU), polyethylene (PE), polyacrylonitrile (PAN), Nylon, acrylonitrile butadiene styrene (ABS), or a combination thereof.

The sealing element 104 is integrally connected on the inner wall of the main body 102, instead of supplied separately. The sealing element 104 is, for instance, a sealing ring. The material of the sealing element 104 is, for instance, a soft material such as a rubber or an elastomer. The elastomer could be a thermoplastic elastomer (TPE), such as thermoplastic polyurethane (TPU), thermoplastic vulcanizate (TPV), or thermoplastic polyolefin (TPO). In an embodiment, the elastomer could be a thermoset elastomer, e.g., silicone, epoxy resin, or a combination thereof.

In the above end cap 100 of the dialyzer and the fabricating method thereof, since the main body 102 and the sealing element 104 are integrally formed in a single piece, the production process and time of the end cap 100 can be effectively reduced.

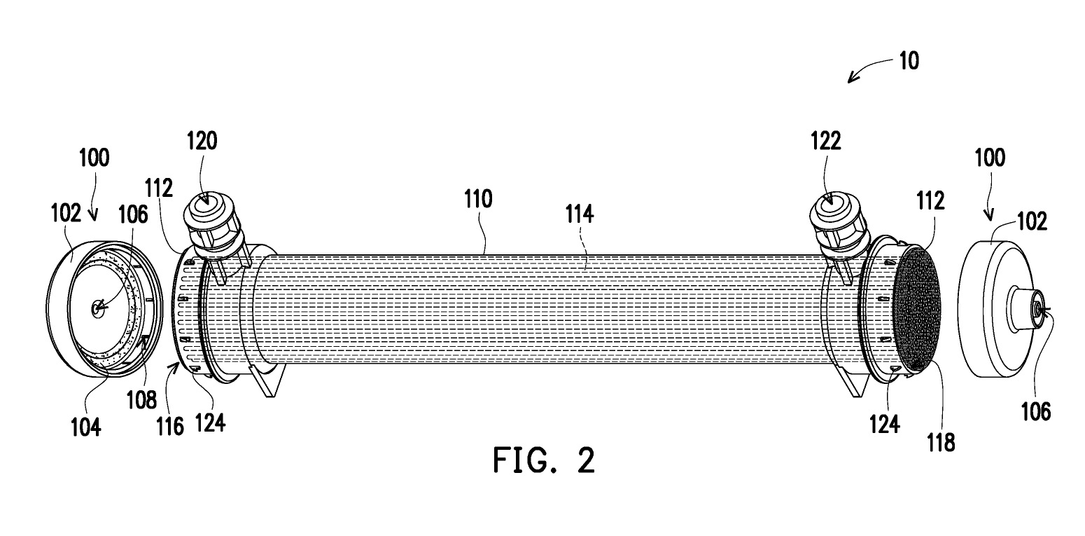

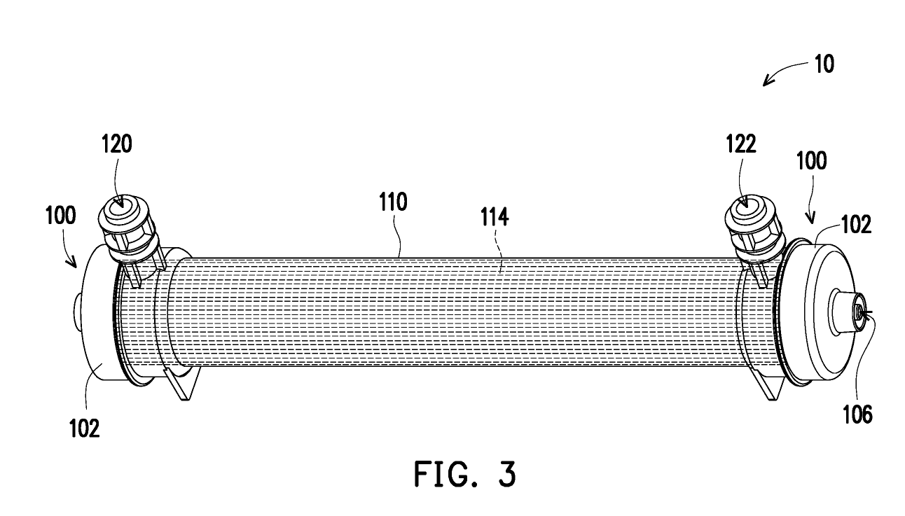

FIG. 2 is a perspective view of a dialyzer according to an embodiment of the invention, of which the housing and the end caps are not combined. FIG. 3 is a perspective view of the dialyzer of FIG. 2, of which the housing and the end caps are combined.

Referring to FIG. 1A, FIG. 1B, FIG. 2, and FIG. 3, a dialyzer 10 includes a housing 110, a sealant 112, a plurality of hollow fiber membranes 114, and two end caps 100. The housing 110 has an opening 116 and an opening 118 opposite to each other that are, for instance, hollow tubular structures housing the hollow fiber membranes 114 therein. A dialysate inlet 120 and a dialysate outlet 122 are disposed on the housing 110, wherein the dialysate inlet 120 is, for instance, close to the opening 116, and the dialysate outlet 122 is, for instance, close to the opening 118. The outer wall of the housing 110 can have at least one latch, snap (e.g., snap hook), notch, or external thread. The housing 110 and the latch, the snap, the notch, or the external thread can be integrally formed. In the present embodiment, the outer wall of the housing 110 is exemplified by having latches 124. The material of the housing 110 is, for instance, polypropylene, polybutylene, polyethylene, or a combination thereof.

The sealant 112 seals the opening 116 and the opening 118 and secures the hollow fiber membranes 114 in the housing 110. Moreover, the dialysate can flow into the housing 110 via the dialysate inlet 120, and flow through a space defined by the sealant 112 located at the two ends of the housing 110, and then flow out of the housing 110 via the dialysate outlet 122. The material of the sealant 120 could be potting compounds such as polyurethane (PU).

The hollow fiber membranes 114 disposed in the housing 110 are secured by the sealant 112. The openings at the two ends of the hollow fiber membranes 114 might not be covered or blocked by the sealant 112, and therefore blood can flow into the hollow fiber membranes 114 through the opening at one end and then flow out through the opening at the other end. The hollow fiber membranes 114 are provided with permeaselectivity and could be semi-permeable membranes. The material of the hollow fiber membranes 114 is, for instance, cellulose acetate, polysulfone (PSU), polyethersulfone (PES), or polymethylmethacrylate (PMMA). In the present embodiment, to increase the compatibility between the hollow fiber membranes 114 and the human body, the hollow fiber membranes 114 can further include a hydrophilic polymer in addition to the main components above. The hydrophilic polymer is, for instance, poly(vinyl pyrrolidone) (PVP), poly(ethylene glycol) (PEG), poly(vinyl alcohol) (PVA), poly(ethylene oxide) (PEO), poly(ethylenimine) (PEI), or poly(acrylate) (PAA). In the present embodiment, the hollow fiber membranes 114 could be prepared by dry-wet spinning. The invention is not limited to the exemplary hollow fiber membranes 114 shown in FIG. 2 and FIG. 3, and thus, those having ordinary skill in the art can adjust the quantity of the hollow fiber membranes 114 as needed. In an embodiment, the quantity of the hollow fiber membranes 114 can be 7000 to 12000.

The end caps 100 respectively cover the two ends of the housing 110. Each of the end caps 100 includes the main body 102 with the blood port 106 and the sealing element 104. The sealing element 104 is integrally connected on the inner wall of the main body 102. Moreover, the two blood ports 106 of the respective end caps 100 can be used as the blood inlet and the blood outlet. In the present embodiment, the blood port 106 as the blood outlet can be set close to the dialysate inlet 120, and the other blood port 106 as the blood inlet can be set close to the dialysate outlet 122, such that the flow directions of the blood and the dialysate in the tube are opposite. As a result, a better dialysis effect can be obtained, but the invention is not limited thereto.

The combining method of the housing 110 and the end caps 100 can include jointing, clipping or screwing. In an embodiment, the housing 110 and the end caps 100 could be combined by engaging the corresponding recess and protrusion structures respectively configured on the housing 110 and end cap 100. When the housing 110 and the end caps 100 are engaged together, assembly time can be reduced, and automated assembly can be facilitated.

For instance, the outer wall of the housing 110 can have a latch, a snap, a notch, or an external thread, and the inner wall of the main body 102 can have a notch, a latch, a snap, or an inner thread. The cross-section shapes of the latch and the notch can be, for instance, polygons (such as triangles or rectangles) or circles, but the invention is not limited thereto, and as long as the cross-section shapes of the latch and the notch are matching and allow the latch and the notch to be clicked together, the shapes are within the scope of the invention.

In an embodiment, when the outer wall of the housing 110 has a latch or a snap, the inner wall of the main body 102 can have a notch corresponding to the latch or the snap of the housing 110, such that the housing 110 and the end caps 100 can be positioned and engaged together. Moreover, when the outer wall of the housing 110 has a notch and the inner wall of the main body 102 has a latch or a snap corresponding to the notch of the housing 110, a similar combining effect can also be achieved.

In another embodiment, when the outer wall of the housing 110 has an external thread, the inner wall of the main body 102 can have an inner thread corresponding to the outer thread of the housing 110, such that the housing 110 and the end caps 100 can be screwed together.

In the present embodiment, the combining method of the housing 110 and the end caps 100 is exemplified by the engagement of the corresponding recesses and protrusions. For instance, the outer wall of the housing 110 can have a latch 124, and the inner wall of the main body 102 can have a notch 108, and therefore the housing 110 and the end caps 100 can be engaged together via the latch 124 and the notch 108.

Moreover, the combining method of the housing 110 and the end caps 100 can include performing ultrasonic welding after the jointing, clipping or screwing between the housing 110 and the end caps 100, so as to further increase the liquid tightness of the dialyzer 10.

The material, fabricating method, and efficacy of the end caps 100 are described in detail in the embodiments above and are therefore not repeated herein. In the dialyzer 10, since the sealing element 104 is accurately positioned and integratedly secured on the main body 102, additional procedure of placing a separate sealing element in a corresponding groove or gap of the end cap can be omitted. As a result, the production steps and production time of the dialyzer 10 can be effectively reduced.

Based on the above, in the end cap of the dialyzer, the fabricating method thereof, and the dialyzer according to the embodiments, the main body and the sealing element are integrally formed in a single piece. Thus, the production process of the end cap and the dialyzer can be simplified, and the production time thereof can be effectively reduced.

Although the invention has been described with reference to the above embodiments, it will be apparent to one of ordinary skill in the art that modifications to the described embodiments may be made without departing from the spirit of the invention. Accordingly, the scope of the invention is defined by the attached claims not by the above detailed descriptions.

What is claimed is:

1. An end cap of a dialyzer, comprising:
a main body having a blood port; and
a sealing element integrally connected on an inner wall of the main body.

2. The end cap of the dialyzer of claim 1, wherein the inner wall of the main body has a notch, a latch, a snap, or an inner thread.

3. The end cap of the dialyzer of claim 1, wherein a material of the main body comprises polyvinylchloride (PVC), polymethylmethacrylate (PMMA), polyethylene terephthalate (PET), polycarbonate (PC), polypropylene (PP), polysulfone (PSU), polyethylene (PE), polyacrylonitrile (PAN), Nylon, acrylonitrile butadiene styrene (ABS), or a combination thereof.

4. The end cap of the dialyzer of claim 1, wherein a material of the sealing element comprises a rubber or an elastomer.

5. The end cap of the dialyzer of claim 4, wherein the elastomer comprises a thermoplastic elastomer or a thermoset elastomer.

6. The end cap of the dialyzer of claim 1, wherein the main body and the sealing element are integrally connected via double injection molding.

7. The end cap of the dialyzer of claim 1, wherein a shrinkage rate difference between the main body and the sealing element is 1% or less.

8. A fabricating method of an end cap of a dialyzer, comprising:
integrally forming a main body and a sealing element using a double injection molding method, wherein
the main body has a blood port,
the sealing element is integrally connected on an inner wall of the main body.

9. The fabricating method of the end cap of the dialyzer of claim 8, wherein the main body is injection-molded before the sealing element is injection-molded, and a softening point of the main body is higher than a softening point of the sealing element.

10. The fabricating method of the end cap of the dialyzer of claim 8, wherein the sealing element is injection-molded before the main body is injection-molded, and a softening point of the sealing element is higher than a softening point of the main body.

11. A dialyzer, comprising:
a housing having two openings opposite to each other, wherein a dialysate inlet and a dialysate outlet are disposed on the housing;
a sealant sealing the two openings;
a plurality of hollow fiber membranes disposed in the housing and secured by the sealant; and
two end caps respectively covering two ends of the housing, wherein each of the end caps comprises:
a main body having a blood port; and
a sealing element integrally connected on an inner wall of the main body.

12. The dialyzer of claim 11, wherein a material of the housing comprises polypropylene (PP), polybutylene, polyethylene (PE), or a combination thereof.

13. The dialyzer of claim 11, wherein a combining method of the housing and the two end caps comprises an engagement of corresponding recess and protrusion structures.

14. The dialyzer of claim 13, wherein an outer wall of the housing has a latch, a snap, a notch, or an outer thread, and the inner wall of the main body correspondingly has a notch, a latch, a snap, or an inner thread.

15. The dialyzer of claim 13, wherein the combining method of the housing and the two end caps further comprises performing ultrasonic welding.

16. The dialyzer of claim 11, wherein a material of the main body comprises polyvinylchloride (PVC), polymethylmethacrylate (PMMA), polyethylene terephthalate (PET), polycarbonate (PC), polypropylene (PP), polysulfone (PSU), polyethylene (PE), polyacrylonitrile (PAN), Nylon, acrylonitrile butadiene styrene (ABS), or a combination thereof.

17. The dialyzer of claim 11, wherein a material of the sealing element comprises a rubber or an elastomer.

18. The dialyzer of claim 17, wherein the elastomer comprises a thermoplastic elastomer or a thermoset elastomer.

19. The dialyzer of claim 11, wherein a forming method of the main body and the sealing element comprises a double injection molding method.

20. The dialyzer of claim 11, wherein a shrinkage rate difference between the main body and the sealing element is 1% or less.
